(19) United States
(12) Patent Application Publication
(10) Pub. No.: US 2025/0044432 A1
(43) Pub. Date: Feb. 6, 2025

(54) ULTRASONIC ELASTICITY IMAGING METHOD AND ULTRASONIC IMAGING APPARATUS

(71) Applicant: SHENZHEN MINDRAY BIO-MEDICAL ELECTRONICS CO., LTD., Shenzhen (CN)

(72) Inventor: Shuangshuang LI, Shenzhen (CN)

(21) Appl. No.: 18/814,383

(22) Filed: Aug. 23, 2024

(30) Foreign Application Priority Data
Aug. 1, 2023 (CN) 202310964274.1

(51) Int. Cl. G01S 7/52

(52) U.S. Cl. CPC G01S 7/52042; G01S 7/52026; G01S 7/52074

(57) ABSTRACT

Disclosed are an ultrasonic elasticity imaging method and an ultrasonic imaging apparatus. The ultrasonic imaging apparatus may determine a stress value according to a first reference value and a second reference value, determine a first quantitative elastic value for a target region according to the stress value and an elastic value measured by strain elastography corresponding to the target region, determine a second quantitative elastic value for a first region of interest according to an elastic value measured by shear wave elastography in the first region of interest and the first quantitative elastic value for the target region, and displaying the second quantitative elastic value.

### CROSS-REFERENCE TO RELATED APPLICATIONS

This application is based on and claims priority to and benefits of Chinese patent Application No. 202310964274.1, filed on Aug. 1, 2023. The entire content of the above-referenced application is incorporated herein by reference.

### TECHNICAL FIELD

The embodiments of the present disclosure relate to medical equipment, and more particularly to ultrasonic elasticity imaging methods and ultrasonic imaging apparatus.

### BACKGROUND OF THE INVENTION

Ultrasonic elastography can non-invasively extract and image the elasticity or softness/hardness information about tissues of a human body, making up for the shortcomings of traditional ultrasonic medical imaging in extraction of mechanical information, and playing an increasingly important role in various clinical practices such as differentiation of malignant from benign tumor, evaluation of the degree of liver fibrosis, angiosclerosis, and muscle and nerve damage.

Shear wave elasticity imaging, a relatively advanced ultrasound elastography technique, may be implemented by generating shear waves inside tissues based on acoustic radiation forces, then detecting their propagation by ultrasound, and calculating physical quantities such as a propagation velocity of the shear waves and a Young's modulus for imaging, thereby quantitatively measuring the elasticity parameters of the tissues and directly representing their hardness. However, due to the fact that shear wave sources generated in tissues are often only at a depth of micrometers, such as only 10 microns, the penetration thereof is limited for large-area and high-hardness lesions (such as malignant cancer), and accurate measurement results cannot be obtained within the lesions. Meanwhile, the spatial resolution of shear wave elasticity images is insufficient to accurately display the hardness, morphology and boundaries of lesions.

### SUMMARY OF THE INVENTION

The embodiments of the present disclosure provide ultrasonic elasticity imaging methods and ultrasonic imaging apparatus for obtaining a quantitative elasticity value for a region where the elastic values measured by shear wave elastography thereof need to be improved, so as to represent the hardness of the tissue corresponding to the region to be improved more accurately.

In one embodiment, there is provided an ultrasonic elasticity imaging method, which may include:
obtaining an elastic value measured by shear wave elastography in a first region of interest (ROI) in a target tissue, and obtaining an elastic value measured by strain elastography in a second region of interest of the target tissue, a tissue region corresponding to the first region of interest and a tissue region corresponding to the second region of interest being at least partially overlapped;
determining a first reference value according to an elastic value measured by shear wave elastography in a first reference region in the first region of interest, and determining a second reference value according to an elastic value measured by strain elastography in a second reference region in the second region of interest;
determining a stress value according to the first reference value and the second reference value;
obtaining a target region where the elastic value measured by shear wave elastography is desired to be improved in the first region of interest, the at least partially overlapped tissue region comprising a tissue region corresponding to the target region;
obtaining an elastic value measured by strain elastography corresponding to the target region from the elastic value measured by strain elastography for the second region of interest, and determining a first quantitative elastic value for the target region according to the stress value and the elastic value measured by strain elastography corresponding to the target region;
determining a second quantitative elastic value for the first region of interest according to the elastic value measured by shear wave elastography for the first region of interest and the first quantitative elastic value for the target region; and
displaying the second quantitative elastic value for the first region of interest.

In one embodiment, said determining a second quantitative elastic value for the first region of interest according to the elastic value measured by shear wave elastography for the first region of interest and the first quantitative elastic value for the target region includes:
replacing the elastic value measured by shear wave elastography for each local point in the target region with the first quantitative elastic value for each local point; the second quantitative elastic value being formed by the first quantitative elastic value for the target region and an elastic value measured by shear wave elastography in a remaining region of the first region of interest excluding the target region.

In one embodiment, said determining a second quantitative elastic value for the first region of interest according to the elastic value measured by shear wave elastography for the first region of interest and the first quantitative elastic value for the target region includes:
for the target region, calculating a weighted sum of an elastic value measured by shear wave elastography in each local point and a first quantitative elastic value for each local point; the second quantitative elastic value being formed by the weighted sum and an elastic value measured by shear wave elastography in a remaining region of the first region of interest excluding the target region.

In one embodiment, the method further includes:
obtaining an elastic value measured by strain elastography in a remaining overlapped region of the at least partially overlapped tissue region excluding the tissue region corresponding to the target region from the elastic value measured by strain elastography for the second region of interest; and
determining a first quantitative elastic value for the remaining overlapped region according to the stress value and the elastic value measured by strain elastography for the remaining overlapped region.

In one embodiment, said determining a second quantitative elastic value for the first region of interest according to the elastic value measured by shear wave elastography for the first region of interest and the first quantitative elastic value for the target region includes:
calculating a weighted sum of an elastic value measured by shear wave elastography in each local point in the target region and a first quantitative elastic value for said each local point;
calculating a weighted sum of an elastic value measured by shear wave elastography in each local point in the remaining overlapped region and a first quantitative elastic value for said each local point;
when the tissue region corresponding to the second region of interest completely contains the tissue region corresponding to the first region of interest, the second quantitative elastic value being formed by the weighted sum of the elastic value measured by shear wave elastography and the first quantitative elastic value for each local point in the target region and the weighted sum of the elastic value measured by shear wave elastography and the first quantitative elastic value for each local point in the remaining overlapped region; and
when the tissue region corresponding to the first region of interest is partially overlapped with the tissue region corresponding to the second region of interest, the second quantitative elastic value being formed by the weighted sum of the elastic value measured by shear wave elastography and the first quantitative elastic value for each local point in the target region, the weighted sum of the elastic value measured by shear wave elastography and the first quantitative elastic value for each local point in the remaining overlapped region, and an elastic value measured by shear wave elastography in a remaining region of the first region of interest excluding the partially overlapped tissue region.

In one embodiment, the target tissue comprises a liver tissue, the target region comprises a focal disease region inside a lesion of the liver tissue, and the remaining overlapped region comprises a diffuse disease region outside the lesion or a non-diseased region outside the lesion of the liver tissue.

In one embodiment, the target tissue includes a liver tissue, the target region comprises a focal disease region inside a lesion of the liver tissue, and the remaining overlapped region comprises a diffuse disease region outside the lesion or a non-diseased region outside the lesion of the liver tissue.

In one embodiment, said determining a first quantitative elastic value for the target region according to the stress value and the elastic value measured by strain elastography corresponding to the target region includes:
for each local point in the target region, calculating a quotient between the stress value and an elastic value measured by strain elastography in said local point, and taking the quotient as the first quantitative elastic value for said local point; and
said determining a stress value according to the first reference value and the second reference value comprises:
calculating a product of the first reference value and the second reference value, and taking the product as the stress value.

In one embodiment, said determining a first reference value according to an elastic value measured by shear wave elastography in a first reference region in the first region of interest includes:
making statistics on a mean of elastic values measured by shear wave elastography for a plurality of local points in the first reference region, and taking the mean as the first reference value; or
determining a median of elastic values measured by shear wave elastography for a plurality of local points in the first reference region, and taking the median as the first reference value.

In one embodiment, said determining a second reference value according to an elastic value measured by strain elastography in a second reference region in the second region of interest includes:
making statistics on a mean of elastic values measured by strain elastography for a plurality of local points in the second reference region, and taking the mean as the second reference value; or
determining a median of elastic values measured by strain elastography for a plurality of local points in the second reference region, and taking the median as the second reference value.

In one embodiment, the first region of interest and the second region of interest correspond to a same tissue region in the target tissue; and
said obtaining an elastic value measured by shear wave elastography in a first region of interest in a target tissue and obtaining an elastic value measured by strain elastography in a second region of interest of the target tissue includes:
transmitting acoustic radiation force impulses to the target tissue to generate shear waves propagating in the same tissue region;
transmitting ultrasonic waves to the same tissue region to track the shear waves propagating in the same tissue region, and receiving echoes of the ultrasonic waves to obtain ultrasonic echo data; and
generating the elastic value measured by shear wave elastography for the first region of interest according to the ultrasonic echo data, and generating the elastic value measured by strain elastography for the second region of interest according to the ultrasonic echo data.

In one embodiment, said obtaining an elastic value measured by shear wave elastography in a first region of interest in a target tissue and obtaining an elastic value measured by strain elastography in a second region of interest of the target tissue includes:
generating first shear waves propagating in the first region of interest;
transmitting first ultrasonic waves to the first region of interest to track the first shear waves propagating in the first region of interest, and receiving echoes of the first ultrasonic waves to obtain first ultrasonic echo signals;
obtaining the elastic value measured by shear wave elastography for the first region of interest according to the first ultrasonic echo signals;
causing a tissue corresponding to the second region of interest to generate displacement or strain;
transmitting second ultrasonic waves at different times to the second region of interest to detect changes in the displacement or strain of the tissue corresponding to the second region of interest, and receiving echoes of the second ultrasonic waves at different times to obtain second ultrasonic echo signals; and
obtaining the elastic value measured by strain elastography for the second region of interest according to the second ultrasonic echo signals.

In one embodiment, the method further includes:
making statistics on second quantitative elastic values for a plurality of local points in the target region, and displaying a statistical result therefrom;
and/or,
making statistics on second quantitative elastic values for a plurality of local points in the first region of interest, and displaying a statistical result therefrom;
the statistical result including a median, a mean, a maximum, a minimum, and a standard deviation.

In one embodiment, the method further includes:
displaying an ultrasonic image corresponding to the target tissue, and displaying on the ultrasonic image a location of the first region of interest and/or a location of the second region of interest; and/or
displaying the elastic value measured by shear wave elastography for the first region of interest; and/or
displaying the elastic value measured by strain elastography for the second region of interest.

In one embodiment, the elastic value measured by shear wave elastography for the first region of interest is represented as a propagation velocity of shear waves, a Young's modulus or a shear modulus; and the first quantitative elastic value is represented as a propagation velocity of shear waves, a Young's modulus or a shear modulus.

In one embodiment, said displaying the second quantitative elastic value for the first region of interest includes:
mapping the second quantitative elastic value for the first region of interest into a quantitative elasticity image with a target display effect, and displaying the quantitative elasticity image with the target display effect; the target display effect including any one or more display effects of grayscale, pseudo-color, and color.

In one embodiment, there is provided an ultrasonic imaging apparatus, which may include:
an ultrasonic probe;
a display;
a transmitting circuit configured to excite the ultrasonic probe to transmit ultrasonic waves to a target tissue of an object under examination;
a receiving circuit configured to control the ultrasonic probe to receive echoes of the ultrasonic waves from the target tissue to obtain ultrasonic echo signals; and
a processor configured to process the ultrasonic echo signals to obtain an ultrasonic image of the target tissue, and
obtain an elastic value measured by shear wave elastography in a first region of interest of a target tissue, and obtain an elastic value measured by strain elastography in a second region of interest of the target tissue, a tissue region corresponding to the first region of interest and a tissue region corresponding to the second region of interest being at least partially overlapped;
determine a first reference value according to an elastic value measured by shear wave elastography in a first reference region in the first region of interest, and determine a second reference value according to an elastic value measured by strain elastography in a second reference region in the second region of interest;
determine a stress value according to the first reference value and the second reference value;
obtain a target region where the elastic value measured by shear wave elastography is desired to be improved in the first region of interest, the at least partially overlapped tissue region comprising a tissue region corresponding to the target region;
obtain an elastic value measured by strain elastography corresponding to the target region from the elastic value measured by strain elastography for the second region of interest, and determine a first quantitative elastic value for the target region according to the stress value and the elastic value measured by strain elastography corresponding to the target region;
determine a second quantitative elastic value for the first region of interest according to the elastic value measured by shear wave elastography for the first region of interest and the first quantitative elastic value for the target region; and
control the display to display the second quantitative elastic value for the first region of interest.

With the present disclosure, a first reference value for a first region of interest and a second reference value for a second reference region may be determined, a stress value may be determined according to the first reference value and the second reference value, a tissue region where the first region of interest and the second region of interest are at least partially overlapped may include a tissue region corresponding to a target region, a first quantitative elastic value for the target region may be determined according to the stress value and an elastic value measured by strain elastography corresponding to the target region, a second quantitative elastic value for the first region of interest may be determined according to an elastic value measured by shear wave elastography in the first region of interest and the first quantitative elastic value for the target region, and the second quantitative elastic value may be displayed. In this way, since the first reference value, the second reference value and the elastic value measured by strain elastography for the target region are accurate, the first and second quantitative elastic values calculated from the above results are more accurate than those obtained by direct shear wave elasticity measurement of the target region, thereby representing the hardness of the tissue corresponding to the target region more accurately and rendering the morphology and boundary of a region corresponding to a lesion with inaccurate elastic value measured by shear wave elastography more accurately.

### BRIEF DESCRIPTION OF THE DRAWINGS

FIG. 1 is a schematic block diagram of the structure of an ultrasonic imaging apparatus in some embodiments of the present disclosure;

FIG. 2 is a schematic flowchart of an ultrasonic elasticity imaging method in some embodiments of the present disclosure;

FIG. 3 is a schematic diagram of an exemplary ultrasonic image on which a first region of interest and an elastic value measured by shear wave elastography thereof are displayed in a display effect in some embodiments of the present disclosure;

FIG. 4 is a schematic diagram of the display of a quantitative elasticity result in a display effect after replacing an elastic value measured by shear wave elastography in the target region shown in FIG. 3 with a first quantitative elastic value;

FIG. 5 is a schematic diagram of an exemplary position relationship between a first region of interest and a second region of interest in some embodiments of the present disclosure; and

FIG. 6 is a schematic diagram of another exemplary position relationship between a first region of interest and a second region of interest in some embodiments of the present disclosure.

### DETAILED DESCRIPTION

The embodiments of the present disclosure provide ultrasonic elasticity imaging methods and ultrasonic imaging apparatus for obtaining a quantitative elasticity result for a region where the elastic values measured by shear wave elastography thereof need to be improved, so as to represent the hardness of the tissue corresponding to the region to be improved more accurately.

The terms “first”, “second”, “third”, “fourth”, etc. (if any) in the specification and claims of the present disclosure and in the drawings attached above are used to distinguish similar objects and are not necessarily used to describe a specific order or sequence. It should be understood that the data used in this way are interchangeable where appropriate so that the embodiments described herein can be implemented in an order other than what is illustrated or described here. In addition, the terms “including” and “having”, as well as any variations thereof, are intended to cover non-exclusive inclusions, for example, a process, method, system, product or device comprising a series of steps or units need not be limited to those steps or units that are clearly listed. Instead, it may include other steps or units that are not clearly listed or are inherent to these processes, methods, products or equipment.

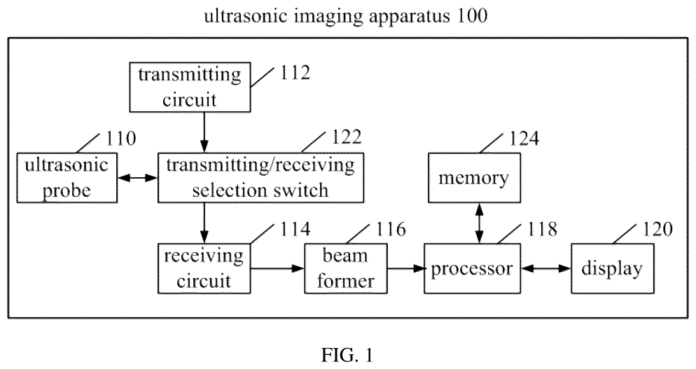

Please refer to FIG. 1, an ultrasonic imaging apparatus 100 in some embodiments may include:
an ultrasonic probe 110, a transmitting circuit 112, a receiving circuit 114, a processor 118 and a display 120. In addition, a transmitting/receiving selection switch 122, a beam former 116 and a memory 124 may also be included.

The ultrasonic probe 110 may be a 2D ultrasonic probe, a 3D ultrasonic probe, or any probe used for ultrasonic inspection and measurement. The acoustic head part of the ultrasonic probe 110 may be an array composed of multiple array elements, such as the array elements arranged in a row to form a line array, or in a two-dimensional matrix to form a plane array. The array elements may also be arranged to form a convex array. The array elements may be used to transmit ultrasonic beams according to excitation of electrical signals, or to convert received ultrasonic echoes into electrical signals. Accordingly, each array element may be used to realize the mutual conversion of the electrical pulse signals and the ultrasonic beams, thereby transmitting ultrasonic waves to a target tissue of a human body and receiving echoes of the ultrasonic waves reflected by the tissue.

The transmitting circuit 112 may be configured to generate transmitting sequences according to the control sent by a transmitting control unit of the processor 118. The transmitting sequences may be configured to control part or all of the array elements to transmit ultrasonic waves to biological tissues.

The receiving circuit 114 may be configured to receive electrical signals of the ultrasonic echoes from the ultrasonic probe 110 to obtain ultrasonic echo signals, and send the ultrasonic echo signals to the beam former 116.

The beam former 116 may be configured to process the signals output by the receiving circuit 114, including delay, weighted summation, and beam forming. Due to the different distances between ultrasonic wave receiving points in the tissue under examination and receiving array elements, the channel data of the same receiving point output from different receiving array elements may have delay differences; accordingly, it is necessary to carry out delay processing to align the phases, weight and sum the different channel data of the same receiving point to obtain beam-formed data.

The processor 118 may be connected with the beam former 116 and may mainly be configured to process, including detecting, signal enhancing, data converting and logarithm compressing, the beam-formed data to generate an ultrasonic image. The ultrasonic image obtained by the processor 118 may be displayed on the display 120 or stored in the memory 124.

Optionally, the processor 118 may be at least one of an application specific integrated circuit (ASIC), a digital signal processor (DSP), a digital signal processing device (DSPD), a programmable logic device (PLD), a field programmable gate array (FPGA), a central processing unit (CPU), a controller, a micro-controller, or a micro-processor, allowing the processor 118 to control other components in the ultrasonic imaging apparatus 100 to perform the ultrasonic imaging steps described in various embodiments disclosed in this specification. The processor 118 may be a component or a general term for control units and processing units in the ultrasonic imaging apparatus capable of controlling other components of the ultrasonic imaging apparatus to perform various functions in various embodiments.

The display 120 may be connected with the processor 118, may be a touch screen or a liquid crystal display, etc. Alternatively, the display 120 may be an independent display such as a liquid crystal display or a television set independent of the ultrasonic imaging apparatus 100. Alternatively, the display 120 may be the display of an electronic device such as a smartphone or tablet, etc. The number of the display 120 may be one or more.

In addition to the above structure, the ultrasonic imaging apparatus 100 may also include a human-machine interaction device. Specifically, the human-machine interaction device may be a display 120, such as all the functions of the human-machine interaction device integrated into the display 120, accordingly, the display 120 can also provide a graphical interface for user interaction when displaying ultrasonic images. One or more controlled objects may be arranged on the graphical interface to provide users with the ability to use the human-machine interaction device to input operation instructions so as to control these controlled objects, thereby executing corresponding control operations. For example, when an icon is displayed on a graphical interface, the icon can be manipulated by a human-machine interaction device to perform specific functions, such as repositioning images and/or zooming in on specific areas.

The human-machine interaction device may also be other human-machine interaction devices besides the display 120. For example, the human-machine interaction device may include an input device for detecting information input by a user. The input information may be such as instructions for editing and marking ultrasonic images, or other types of instructions. The input device may include a keyboard, a scroll wheel, a trackball, and a mobile input device (such as a mobile device with a touch screen, a cell phone, etc.), a multifunctional knob, and any combination thereof. The human-machine interaction device may also include an output device such as a printer.

The ultrasonic imaging apparatus 100 mentioned above may also include a memory 124 for storing instructions for processing execution, for storing received ultrasonic echo signals, for storing ultrasonic image data, and so on. The memory 124 may be a volatile memory, such as a random access memory (RAM), or be a non-volatile memory, such as a read only memory (ROM), a flash memory, a hard disk drive (HDD), or a solid state drive (SSD); or a combination thereof; and may provide instructions and data to the processor.

It should be understood that the components included in the ultrasonic imaging apparatus 100 shown in FIG. 1 are illustrative only and may include more or fewer components, which are not limited in the present application.

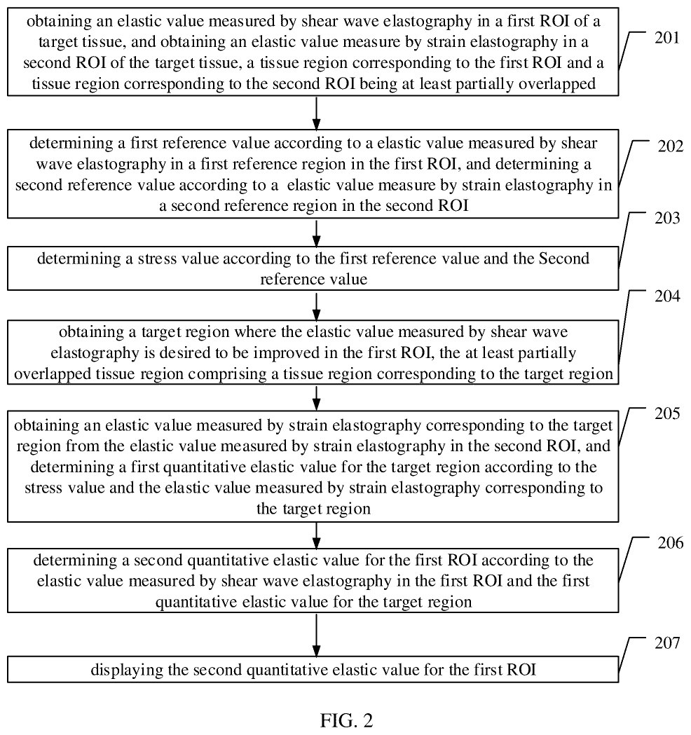

The embodiments and implementations thereof of an ultrasonic elasticity imaging method executed by the ultrasonic imaging apparatus may further be described in detail below according to the specific structural components and functions thereof of the ultrasonic imaging apparatus mentioned above. Please refer to FIG. 2, the ultrasonic elasticity imaging method in some embodiments of the present disclosure may include the following steps:

Step 201: obtaining an elastic value measured by shear wave elastography in a first region of interest in a target tissue, and obtaining an elastic value measured by strain elastography in a second region of interest of the target tissue, a tissue region corresponding to the first region of interest and a tissue region corresponding to the second region of interest being at least partially overlapped;

The target tissue may be the liver, spleen, pancreas, kidney and other tissues of an object under examination, and may be performed with ultrasound scanning with the ultrasonic probe. In this respect, the ultrasonic imaging apparatus may, with the ultrasonic probe, transmit ultrasonic waves to the target tissue of the object under examination, and receive the echoes of the ultrasonic waves to obtain ultrasonic echo signals according to the echoes of the ultrasonic waves. The ultrasonic echo signals may be configured to generate the ultrasonic image of the target tissue. The ultrasonic elasticity imaging method and the ultrasonic imaging apparatus provided in the present disclosure may be applied to a human body and various animals, that is, the object under examination may be a human body or various animals.

The first region of interest of the target tissue may be performed with shear wave elasticity measurement to obtain the elastic value measured by shear wave elastography for the first region of interest; and the second region of interest of the target tissue may be performed with strain elasticity measurement to obtain the elastic value measured by strain elastography for the second region of interest. The tissue region corresponding to the first region of interest and the tissue region corresponding to the second region of interest may be at least partially overlapped, that is, the first region of interest and the second region of interest may include the same tissue region.

Step 202: determining a first reference value according to an elastic value measured by shear wave elastography in a first reference region in the first region of interest, and determining a second reference value according to an elastic value measured by strain elastography in a second reference region in the second region of interest;

Step 203: determining a stress value according to the first reference value and the second reference value;

There may be part of the first region of interest that the depth thereof in the target tissue is deep and the hardness of the tissue corresponding thereto is high, i.e., a tissue region with deep depth and high hardness in the target tissue. Due to its characteristics of deep depth and high hardness, the penetration of shear waves at this tissue region may be weakened, leading to an inaccurate elastic value measured by shear wave elastography therefor, and affecting the diagnosis of shear wave elasticity measurement. To improve the measurement of such tissue region to represent the hardness of the tissue more accurately, in some embodiments, a first reference value may be determined according to an elastic value measured by shear wave elastography in a first reference region in the first region of interest, the elastic value measured by shear wave elastography for the first reference region may be more accurate compared with the aforesaid elastic value measured by shear wave elastography for the tissue region with deep depth and high hardness in the target tissue, and accordingly the calculated first reference value may also be accurate. For example, the elastic value measured by shear wave elastography for a tissue region with shallow depth or a non-diseased region in the target tissue may be used to calculate the first reference value.

In addition, the second reference value may be determined according to an elastic value measured by strain elastography in a second reference region in the second region of interest. Since the depth, hardness and other factors of tissues have relatively little influence on the accuracy of strain elasticity measurement, the second reference region may be any region in the second region of interest, and the elastic value measured by strain elastography for the second reference region may be accurate, as well as the calculated second reference value.

The stress value may be determined according to the first reference value and the second reference value, and may be configured to improve the inaccurate elastic value measured by shear wave elastography for some tissue regions to obtain an accurate measurement result.

Step 204: obtaining a target region where the elastic value measured by shear wave elastography is desired to be improved in the first region of interest, the at least partially overlapped tissue region including the tissue region corresponding to the target region;

Step 205: obtaining an elastic value measured by strain elastography corresponding to the target region from the elastic value measured by strain elastography for the second region of interest, and determining a first quantitative elastic value for the target region according to the stress value and the elastic value measured by strain elastography corresponding to the target region;

The target region may be a region where the elastic value measured by shear wave elastography is inaccurate in the target tissue due to its characteristics of deep depth and high hardness, such as the focal lesion region of the target tissue which has relatively high hardness and inaccurate elastic value measured by shear wave elastography obtained. In addition, the target region where the elastic value measured by shear wave elastography thereof is desired to be improved may be determined from the shear wave elasticity image of the first region of interest by doctors based on their own experience in measuring shear wave elasticity. The tissue region where the first region of interest and the second region of interest are at least partially overlapped may include the tissue region corresponding to the target region.

The stress corresponding to each point in a tissue region where strain elasticity measurement is performed is generally the same or approximately constant; that is, for the second region of interest, the stress to which each point is subjected during strain elasticity measurement is generally the same or approximately constant. Based on this, the elastic value measured by strain elastography corresponding to the target region may be obtained from the elastic value measured by strain elastography for the second region of interest. Since the stress to which each point in the second region of interest is subjected is the same or approximately unchanged and the second region of interest includes the target region, the first quantitative elastic value for the target region may be determined according to the stress value and the elastic value measured by strain elastography corresponding to the target region that are calculated in the above steps.

Step 206: determining a second quantitative elastic value for the first region of interest according to the elastic value measured by shear wave elastography for the first region of interest and the first quantitative elastic value for the target region; and

Step 207: displaying the second quantitative elastic value for the first region of interest.

After calculating the first quantitative elastic value for the target region, the second quantitative elastic value for the first region of interest may be determined according to the elastic value measured by shear wave elastography for the first region of interest and the first quantitative elastic value for the target region, and the second quantitative elastic value for the first region of interest may be displayed.

In some embodiments of the present disclosure, the ultrasonic imaging apparatus may determine the first reference value for the first region of interest and the second reference value for the second reference region, determine the stress value according to the first reference value and the second reference value, determine the first quantitative elastic value for the target region (wherein the tissue region corresponding to the target region is included in the tissue region where the first region of interest and the second region of interest are at least partially overlapped) according to the stress value and the elastic value measured by strain elastography corresponding to the target region, determine the second quantitative elastic value for the first region of interest according to the elastic value measured by shear wave elastography for the first region of interest and the first quantitative elastic value for the target region, and display the second quantitative elastic value. In this way, since the first reference value, the second reference value and the elastic value measured by strain elastography for the target region are accurate, the first quantitative elastic value and the second quantitative elastic value that are obtained by the calculation according to the above results can be more accurate compared with the result obtained by directly measuring the shear wave elasticity of the target region; thereby representing the hardness of the tissue corresponding to the target region more accurately, and rendering the morphology and boundary of the region (such as a lesion) with inaccurate elastic value measured by shear wave elastography more accurately.

The ultrasonic imaging apparatus may generate an ultrasonic image by performing ultrasound scanning on the target tissue with the ultrasonic probe handheld by a user, determine the first region of interest in the ultrasonic image, and perform shear wave elasticity measurement on the first region of interest to obtain the elastic value measured by shear wave elastography; or determine the second region of interest in the ultrasonic image and perform strain elasticity measurement on the second region of interest to obtain the elastic value measured by strain elastography. In some embodiments, the ultrasonic image used for obtaining the elastic value measured by shear wave elastography for the first region of interest and the ultrasonic image used for obtaining the elastic value measured by strain elastography for the second region of interest may be obtained by imaging the same section or position of the target tissue. In addition, the above two ultrasonic images may also be obtained by imaging different sections or positions of the target tissue, but the above two ultrasonic images should be obtained by imaging the same target region in the target tissue, that is, the above two ultrasonic images should contain the same target region.

The first region of interest and the second region of interest may be at least partially overlapped, and the at least partially overlapped tissue region may include the tissue region corresponding to the target region.

In some embodiments, the ultrasonic image used for obtaining the elastic value measured by shear wave elastography for the first region of interest and the ultrasonic image used for obtaining the elastic value measured by strain elastography for the second region of interest may be directed at the same section or position of the target tissue, and the first region of interest and the second region of interest may be completely overlapped. This can be achieved by manually adjusting the position and angle of the probe, or by fixing the probe, or by based on cross-sectional matching, and so on.

Another way to achieve the above effects may be that the first region of interest and the second region of interest may correspond to the same tissue region in the target tissue; and the ultrasonic imaging apparatus may transmit acoustic radiation force impulses to the target tissue to generate shear waves propagating in the same tissue region, transmit ultrasonic waves to the same tissue region to track the shear waves propagating in the same tissue region, receive the echoes of the ultrasonic waves to obtain ultrasonic echo data, and generate the elastic value measured by shear wave elastography for the first region of interest, as well as the elastic value measured by strain elastography for the second region of interest, according to the ultrasonic echo data.

In this way, the elastic value measured by shear wave elastography and the elastic value measured by strain elastography may be obtained simultaneously according to the same ultrasonic image and the same region of interest, with the section corresponding to the ultrasonic image being completely consistent and the region of interest being completely consistent.

Moreover, for this method, compared to obtaining the elastic value measured by shear wave elastography for the first region of interest by shear wave elasticity measurement and obtaining the elastic value measured by strain elastography for the second region of interest by strain elasticity measurement separately, it is unnecessary to switch the measurement modes (such as switching from shear wave elasticity measurement to strain elasticity measurement, or from strain elasticity measurement to shear wave elasticity measurement), simplifying user operations and improving the efficiency of obtaining the elastic value measured by shear wave elastography and the elastic value measured by strain elastography.

The above is the wave elasticity result for the first region of interest and the elastic value measured by strain elastography for the second region of interest obtained simultaneously in a duplex mode; however, they may also be obtained sequentially in a non-duplex mode. In the non-duplex mode, the ultrasonic imaging apparatus may generate first shear waves propagating in the first region of interest, transmit first ultrasonic waves to the first region of interest to track the first shear waves propagating in the first region of interest, and receive the echoes of the first ultrasonic waves to obtain first ultrasonic echo signals, thus obtaining the elastic value measured by shear wave elastography for the first region of interest according to the first ultrasonic echo signals. The ultrasonic imaging apparatus may also cause the tissue corresponding to the second region of interest to generate displacement or strain, transmit second ultrasonic waves at different times to the second region of interest to track changes in the displacement and strain of the tissue corresponding to the second region of interest, receive the echoes of the second ultrasonic waves at different times to obtain second ultrasonic echo signals, and obtain the elastic value measured by strain elastography for the second region of interest according to the second ultrasonic echo signals.

The way to cause the tissue corresponding to the second region of interest to generate displacement or strain may be according to mechanical force (such as pressing the part corresponding to target tissue), or may be according to the acoustic radiation force impulses. In the non-duplex mode, the sequence of performing shear wave elasticity measurement and strain elasticity measurement is not limited in some or all embodiments.

In some embodiments, when determining the second quantitative elastic value for the first region of interest according to the elastic value measured by shear wave elastography for the first region of interest and the first quantitative elastic value for the target region, the elastic value measured by shear wave elastography for each local point in the target region may be replaced with the first quantitative elastic value for the each local point, and in this respect, the second quantitative elastic value for the first region of interest may be formed by the first quantitative elastic value for the target region and the elastic value measured by shear wave elastography for the remaining region of the first region of interest excluding the target region.

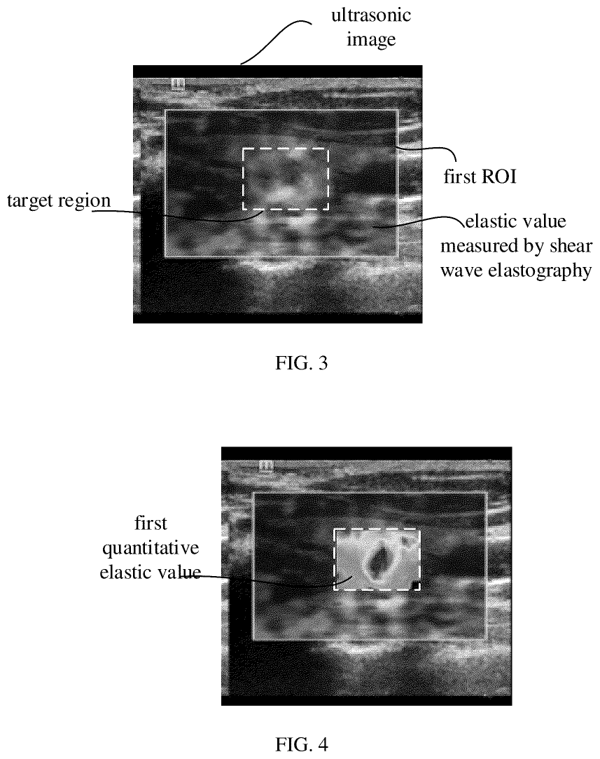

As shown in FIG. 3, after obtaining the ultrasonic image of the target tissue, a first region of interest thereof (the region marked by a rectangular box in the ultrasonic image shown in FIG. 3) may be determined, the elastic value measured by shear wave elastography may be obtained by performing shear wave elasticity measurement on the first region of interest, and the target region where the shear wave elasticity thereof is desired to be improved in the first region of interest may be determined. After obtaining the first quantitative elastic value for the target region, the elastic value measured by shear wave elastography for each local point in the target region may be replaced with the first quantitative elastic value for said each local point, that is, the target region may be displayed as the first quantitative elastic value shown in FIG. 4. Then the elastic value measured by shear wave elastography and the first quantitative elastic value for the first region of interest shown in FIG. 4 may form the second quantitative elastic value.

In addition, after replacing the elastic value measured by shear wave elastography for each local point in the target region with the first quantitative elastic value for each local point, there may be an obvious transition trace between the data at the edge of the target region and the elastic value measured by shear wave elastography for the surrounding region thereof. In this respect, image smoothing, weighting, median filtering and other processing methods may be used to process the edge data to reduce the transition trace at the boundary.

When determining the second quantitative elastic value for the first region of interest according to the elastic value measured by shear wave elastography for the first region of interest and the first quantitative elastic value for the target region, in some embodiments, the weighted sum of the elastic value measured by shear wave elastography for each local point in the target region and the first quantitative elastic value for said each local point may be calculated. In this respect, the second quantitative elastic value may be formed by the weighted sum corresponding to each local point in the target region and the elastic value measured by shear wave elastography for the remaining region of the first region of interest excluding the target region.

For example, according to the elastic value measured by shear wave elastography for the target region shown in FIG. 3, after obtaining the first quantitative elastic value for the target region, the weighted sum of the elastic value measured by shear wave elastography for each local point in the target region and the first quantitative elastic value for said each local point may be calculated, and the second quantitative elastic value may be formed by the weighted sum corresponding to the local point in the target region and the elastic value measured by shear wave elastography for the remaining region of the first region of interest excluding the target region. When calculating the weighted sum, the sum of a weight corresponding to the elastic value measured by shear wave elastography for each local point in the target region and a weight corresponding to the first quantitative elastic value for said each local point may be any numerical value. For example, the sum of the weights may be 1, 100, or any other numerical value, without limitation here. In addition, the two weights may also be randomly set by users, which may be determined according to the reliability or accuracy of the elastic value measured by shear wave elastography for the target region. When the reliability or accuracy are low, the weight corresponding to the elastic value measured by shear wave elastography for the local point in the target region may be set to be small.

In some embodiments, besides the calculation of the first quantitative elastic value for the target region, the elastic value measured by strain elastography for the remaining overlapped region in the at least partially overlapped tissue region excluding the tissue region corresponding to the target region may be obtained from the elastic value measured by strain elastography for the second region of interest, and the first quantitative elastic value for the remaining overlapped region may be determined according to the stress value and the elastic value measured by strain elastography for the remaining overlapped region that are obtained by the above calculation.

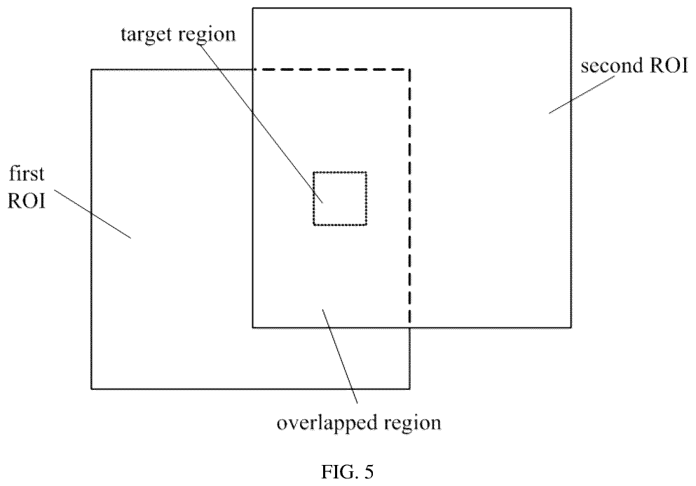

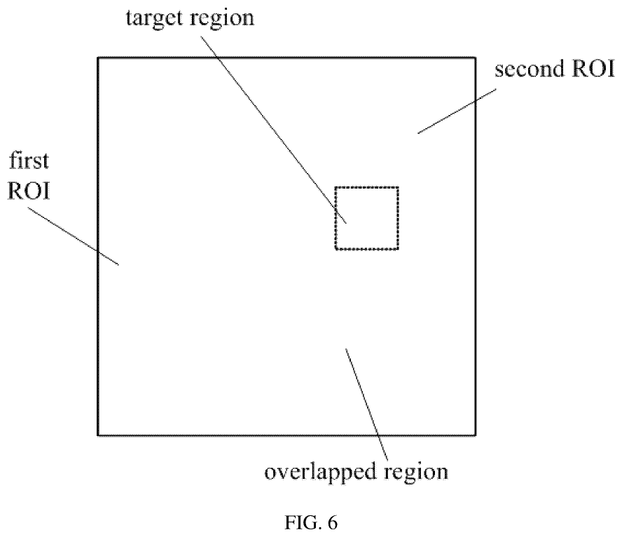

For example, as shown in FIG. 5, there may be an overlapped region between the first region of interest and the second region of interest, and the overlapped region may include the target region where the elastic value measured by shear wave elastography thereof is desired to be improved. Besides the calculation of the first quantitative elastic value for the target region, the first quantitative elastic value for the remaining overlapped region excluding the target region may also be calculated.

It should be noted that the shapes and locations of various regions shown in FIG. 5 are only illustrative and do not limit the scope of the shown embodiments. It may only define in some embodiments that the tissue regions each corresponding to the first region of interest and the second region of interest may be at least partially overlapped, and that the at least partially overlapped tissue region may include the tissue region corresponding to the target region. The shapes of the various regions mentioned above are not limited.

Further, when determining the second quantitative elastic value for the first region of interest according to the elastic value measured by shear wave elastography for the first region of interest and the first quantitative elastic value for the target region, in some embodiments, the weighted sum of the elastic value measured by shear wave elastography for each local point in the target region and the first quantitative elastic value for said each local point may be calculated; and the weighted sum of the elastic value measured by shear wave elastography for each local point in the remaining overlapped region and the first quantitative elastic value for said each local point may be calculated.

Accordingly, when the tissue region corresponding to the first region of interest is partially overlapped with the tissue region corresponding to the second region of interest, the second quantitative elastic value may be formed by the weighted sum corresponding to the local point in the target region, the weighted sum corresponding to the local point in the remaining overlapped region, and the elastic value measured by shear wave elastography for the rest other than the at least partially overlapped tissue region in the first region of interest (i.e. the region that does not overlap with the second region of interest). As shown in FIG. 5, the second quantitative elastic value may be formed by the elastic value measured by shear wave elastography for a region where the first region of interest and the second region of interest are not overlapped, the first quantitative elastic value for the target region, and the first quantitative elastic value for the remaining overlapped region excluding the target region.

When the tissue region corresponding to the second region of interest completely includes the tissue region corresponding to the first region of interest, the second quantitative elastic value may be formed by the weighted sum corresponding to the local point in the target region and the weighted sum corresponding to the local point in the remaining overlapped region. As shown in FIG. 6, when the first region of interest and the second region of interest are completely overlapped, the second quantitative elastic value may be formed by the first quantitative elastic value for the target region and the first quantitative elastic value for the remaining overlapped region excluding the target region. FIG. 6 is only an example. The first region of interest and the second region of interest may not completely overlap, as long as the tissue region corresponding to the second region of interest can fully contain the tissue region corresponding to the first region of interest. The second region of interest and the first region of interest may be displayed in the same window or different windows, which is not limited here.

For the target region, it is assumed that the weight corresponding to the elastic value measured by shear wave elastography for the local point is a; and for the remaining overlapped region excluding the target region, it is assumed that the weight corresponding to the elastic value measured by shear wave elastography for the local point is B; then it may be α=β or α≠β.

For example, if the sum of the weights is 1, the weight corresponding to the elastic value measured by shear wave elastography for the local point in the target region is α, the weight corresponding to the first quantitative elastic value for the local point in the target region is 1-α, where 0<α<1; and the weight corresponding to the elastic value measured by shear wave elastography for the local point in the remaining overlapped region excluding the target region is β, the weight corresponding to the first quantitative elastic value for the local point in the remaining overlapped region excluding the target region is 1-β, where 0<β<1. α and β may be equal or unequal, which can be set independently by users. For example, if it is believed that the elastic value measured by shear wave elastography for the remaining overlapped region is more reliable and accurate, a larger value of β may be set; while if it is believed that the elastic value measured by shear wave elastography for the target region is less reliable and accurate, a smaller value of α may be set, and the weight of the first quantitative elastic value for the target region may be larger.

Similarly, after calculating the weighted sum corresponding to the target region or the weighted sum corresponding to the remaining overlapped region, there may be an obvious transition trace between the data at the edge of the target region and the data of the region therearound. In this respect, image smoothing, weighting, median filtering and other processing methods may also be used to process the edge data to reduce the transition trace at the border.

In some embodiments, the target tissue may be any parenchymal tissue, such as liver tissue, kidney tissue, spleen tissue, pancreas tissue, and so on. When the target tissue is a liver tissue, the target region may include a focal disease region inside a lesion in the liver tissue, the penetration of shear waves at such region is limited, accordingly it is difficult to obtain accurate elastic values measured by shear wave elastography. The remaining overlapped region in the first and second regions of interest excluding the target region may include a diffuse disease region outside the lesion or a non-diseased region outside the lesion of the liver tissue, the penetration of shear waves at such region is less restricted, accordingly elastic values measured by shear wave elastography can be obtained accurately.

In this way, with the above embodiments, the first quantitative elastic value for the lesion region of a tissue may be obtained, which may be more accurate and reliable than that obtained by conventional shear wave elasticity measurement. Accordingly, the hardness of the lesion region may be represented more accurately, and the hardness, morphology and boundary of the lesion can be rendered more accurately according to the first quantitative elastic value.

The elastic value measured by shear wave elastography may include the Young's modulus result. According to Hooke's law, under a certain stress Stress, the elastic value measured by strain elastography Strain is inversely proportional to the Young's modulus result E, which can be expressed by a formula Stress=Strain*E. The stress corresponding to each point in a tissue region where strain elasticity measurement is performed is generally the same or approximately constant; that is, the stress to which each point in the second region of interest is subjected during the strain elasticity measurement is generally the same or approximately constant. Based on this, in some embodiments, according to the formula Stress=Strain*E, the product of the first reference value and the second reference value may be calculated. The product is the stress value, which may represent the stress to which each point in the second region of interest is subjected. Further, according to the formula Stress=Strain*E, for each local point in the target region, a quotient between the stress value and the elastic value measured by strain elastography for each local point may be calculated. The quotient may be taken as the first quantitative elastic value for each local point, and the first quantitative elastic value may be represented as the Young's modulus result E.

The calculated first quantitative elastic value is the Young's modulus result E, but the Young's modulus result E may also be further converted into physical quantities that represent the elastic value measured by shear wave elastography such as the propagation velocity of shear waves, the shear modulus and so on. Accordingly, the elastic value measured by shear wave elastography for the first region of interest may be represented as the propagation velocity of shear waves, Young's modulus or shear modulus; and the first quantitative elastic value may also be represented as the propagation velocity of shear waves, Young's modulus or shear modulus.

In some embodiments, when the rest other than the target region in the first region of interest is the non-diseased region or the diffuse disease region outside the lesion, the elastic value measured by shear wave elastography for the rest region are accurate and reliable. In this respect, the elastic values measured by shear wave elastography for individual points in the rest region may be also distributed evenly, and the first reference values corresponding to different regions in the rest region may be the same or approximately the same. Further, the first reference region may be any region in the rest region, and the first reference region and the second reference region may be different regions. In addition, in some embodiments, the first reference region and the second reference region may be overlapped partially or completely.

For example, the first reference region and the second reference region being overlapped partially or completely may be that both the first and second reference regions are located in the overlapped region of the first and second regions of interest, and the first and second reference regions may be partially or completely overlapped with each other.

The calculation of the first reference value may be implemented by making statistics on the mean of the elastic values measured by shear wave elastography for a plurality of local points in the first reference region and taking the mean as the first reference value; or by determining the median of the elastic values measured by shear wave elastography for a plurality of local points in the first reference region and taking the median as the first reference value.

Besides the calculation of the mean or median of elastic values measured by shear wave elastography, other statistical physical quantities that can represent the average level of the elastic value measured by shear wave elastography for the first reference region may also be calculated, such as the mode of the elastic values measured by shear wave elastography for a plurality of local points in the first reference region, and so on.

Similarly, the determination of the second reference value may be implemented by making statistics on the mean of the elastic values measured by strain elastography for a plurality of local points in the second reference region and taking the mean as the second reference value; or by determining the median of the elastic values measured by strain elastography for a plurality of local points in the second reference region and taking the median as the second reference value.

In addition to calculating the mean or median of elastic values measured by strain elastography, other statistical physical quantities that can represent the average level of the elastic value measured by strain elastography for the second reference region may also be calculated, such as the mode of the elastic values measured by strain elastography for a plurality of local points in the second reference region, and so on.

In some embodiments, after determining the second quantitative elastic value for the first region of interest, it may make statistics on the second quantitative elastic values for a plurality of local points in the target region and the statistical result therefrom may be displayed. The statistical result may include any one or more of the median, mean, maximum, minimum, and standard deviation. In this way, with the statistical result, the hardness distribution of the tissue region corresponding to the target region may be known. For example, users may know the difference in the hardness distribution of the tissue region corresponding to the target region by the standard deviation, the local point with the minimum or maximum hardness in the tissue region by the minimum or the maximum, the overall hardness of the tissue region by the mean, and so on.

In addition, the second quantitative elastic values for a plurality of local points in the first region of interest may be made statistics for the whole first region of interest, and the statistical result therefrom may be displayed. The statistical result may include one or more of median, mean, maximum, minimum, and standard deviation. Similarly, with the statistical result, the hardness distribution of the tissue region corresponding to the first region of interest may be known. For example, users may know the difference in the hardness distribution of the tissue region corresponding to the first region of interest by the standard deviation, the local point with the minimum or maximum hardness in the tissue region by the minimum or the maximum, the overall hardness of the tissue region by the mean, and so on.

In some embodiments, besides the display of the second quantitative elastic value for the first region of interest, the ultrasonic imaging apparatus may also display the ultrasonic image corresponding to the target tissue, wherein the second quantitative elastic value for the first region of interest may be displayed in a location corresponding to the first region of interest in the ultrasonic image, as shown in FIG. 4.

In addition, the location of the first region of interest and/or the location of the second region of interest may be displayed on the ultrasonic image; for example, the location of the first region of interest and/or the location of the second region of interest may be shown and circled in a closed graphic of any shape. As shown in FIG. 3 or FIG. 4, the location of the first region of interest may be shown in a box to allow users to differentiate the location of the first region of interest from the location of the second region of interest.

When displaying the second quantitative elastic value for the first region of interest, the elastic value measured by shear wave elastography for the first region of interest may also be displayed, facilitating users to compare the result obtained by conventional shear wave elasticity measurement and the second quantitative elastic value obtained by the above quantitative calculation, and to compare the differences in hardness and elasticity represented by the two results.

In addition, the elastic value measured by strain elastography for the second region of interest may also be displayed to facilitate user to compare the differences in tissue hardness and elasticity obtained by the strain elasticity measurement and by the above quantitative calculation.

In some embodiments, when displaying the second quantitative elastic value for the first region of interest, the second quantitative elastic value for the first region of interest may be mapped into the quantitative elasticity image with a target display effect, and the quantitative elasticity image of the target display effect may be displayed. The target display effect may include any one or more display effects of grayscale, pseudo-color, and color.

As shown in FIG. 4, after replacing the elastic value measured by shear wave elastography for the target region with the first quantitative elastic value, the first quantitative elastic value may be mapped into the quantitative elasticity image that is in any of grayscale, pseudo-color, and color; and the elastic value measured by shear wave elastography for the rest other than the target region in the first region of interest may also be mapped into an elasticity image that is in any of grayscale, pseudo-color, and color. The quantitative elasticity image corresponding to the target region and the elasticity image corresponding to the rest region may form the quantitative elasticity image of the second quantitative elastic value which may be displayed at a location corresponding to the first region of interest in the ultrasonic image.

The conventional strain elasticity image can only qualitatively reflect the hardness of tissues; for example, a red color block in a strain elasticity image may represent that the hardness of the tissue region corresponding thereto is relatively high, and a blue color block may represent that the hardness of the tissue region corresponding thereto is relatively low. In some embodiments, the first quantitative elastic value and the second quantitative elastic value may be obtained by the quantitative calculation according to the elastic value measured by strain elastography, and the first quantitative elastic value and the second quantitative elastic value may quantitatively reflect the hardness of tissues, such as different colors representing different Young's moduli, thereby quantitatively representing the hardness of the tissue region.

In some embodiments, the first region of interest, the second region of interest, the target region, the first reference region and the second reference region may be obtained automatically by the ultrasonic imaging apparatus. The ultrasonic imaging apparatus may determine the aforesaid regions based on preset rules. For example, if a preset rule indicates selecting a region containing lesion as the first region of interest, the ultrasonic imaging apparatus may automatically determine the first region of interest according to this rule; or it may automatically determine the target region in the first region of interest according to the reliability or quality of the elastic value measured by shear wave elastography. In addition, the above regions may also be manually determined by users, that is, operations for setting the regions may be inputted by users, and the ultrasonic imaging apparatus may determine the regions based on the setting operations. The way to determine the regions is not limited herein.

Therefore, through the above embodiments and various preferred implementations of the present disclosure, it is possible to correct the less accurate and reliable elastic values measured by shear wave elastography obtained by conventional shear wave elastic measurements, and to calculate quantitative elasticity results according to the first reference values and second reference values with high accuracy and reliability. Compared with the conventional shear wave elasticity measurements, the results obtained by the method or apparatus disclosed herein are more accurate and more reliable, so it can more accurately represent the tissue hardness and elasticity of the region with inaccurate elastic values measured by shear wave elastography such as lesions, and better render the morphology and boundary of the region with inaccurate elastic values measured by shear wave elastography such as lesions.

Below, based on the specific structural components and functions of the ultrasonic imaging apparatus shown in FIG. 1, the various components of the ultrasonic imaging apparatus, as well as the functions and operations performed by each component thereof, will be further explained in detailed.

In some embodiments, the ultrasonic imaging apparatus may include:
an ultrasonic probe;
a transmitting circuit for exciting the ultrasonic probe to transmit ultrasonic waves to a target tissue of an object under examination;
a receiving circuit for controlling the ultrasonic probe to receive echoes of the ultrasonic waves from the target tissue to obtain ultrasonic echo signals; and
a processor for processing the ultrasonic echo signals to obtain an ultrasonic image of the target tissue and, according to the ultrasonic image, performing the ultrasonic elasticity imaging method executed by the ultrasonic imaging apparatus in the above embodiments shown in FIG. 2 and the implementations thereof.

The functions and operations performed by the components of the ultrasonic imaging apparatus in some embodiment are similar to those performed by the ultrasonic imaging apparatus in the embodiments shown in FIG. 2 above and their multiple preferred implementations, and will not be repeated here.

In some embodiments of the present disclosure, the ultrasonic imaging apparatus may determine the first reference value for the first region of interest and the second reference value for the second reference region, determine the stress value according to the first reference value and the second reference value, determine the first quantitative elastic value for the target region (wherein the tissue region corresponding to the target region is included in the tissue region where the first region of interest and the second region of interest are at least partially overlapped) according to the stress value and the elastic value measured by strain elastography corresponding to the target region, determine the second quantitative elastic value for the first region of interest according to the elastic value measured by shear wave elastography for the first region of interest and the first quantitative elastic value for the target region, and display the second quantitative elastic value. In this way, since the first reference value, the second reference value and the elastic value measured by strain elastography for the target region are accurate, the first quantitative elastic value for the target region that are obtained by the calculation according to the above results can be more accurate compared with the result obtained by directly measuring the shear wave elasticity of the target region; thereby rendering the morphology and boundary of the region (such as a lesion) with inaccurate elastic value measured by shear wave elastography more accurately.

Those skilled in the art can clearly understand that, for the sake of convenience and simplicity in description, the specific working process of the system, device and unit described above can refer to the corresponding process in the above-mentioned embodiments of the method, and will not be repeated here.

In a plurality of embodiments provided in the present disclosure, it should be understood that the disclosed systems, apparatus, and methods may be implemented in other ways. For example, the device embodiments described above are only illustrative. For example, the division of units is only a logical functional division. In practical implementations, there may be other division methods, such as multiple units or components being combined or integrated into another system, or some features being ignored or not executed. In addition, the coupling or direct coupling or communication connection displayed or discussed may be indirect coupling or communication connection through some interface, device or unit, which may be electrical, mechanical or other forms.

The units described as separate components may or may not be physically separate, and the components displayed as units may or may not be physical units, that is, they may be located in one place, or may be distributed across multiple network units. Some or all of the units may be selected according to the actual needs to achieve the purpose of the embodiments.

In addition, the functional units in various embodiments of the present disclosure may be integrated in a single processing unit, or the units may exist separately physically, or two or more units may be integrated in a single unit. The integrated unit mentioned above may be realized either in the form of hardware or in the form of software functional unit.

The integrated unit, if implemented in the form of software functional units and marketed or used as an independent product, may be stored in a computer readable storage medium. Based on this understanding, the technical solution of the present disclosure may be substantially or partially contributed to the prior art or all or part of the technical solution may be embodied in the form of a software product, which is stored in a storage medium and includes a number of instructions for making a computer device (which may be a personal computer, a server, or a network device, etc.) perform all or part of the steps of the method described in various embodiments of the present disclosure. The aforementioned storage media may include: USB disk, mobile hard disk, read-only memory (ROM), random access memory (RAM), disk or disc and other media that can store program code.

What is claimed is:

1. An ultrasonic elasticity imaging method, comprising:
obtaining an elastic value measured by shear wave elastography in a first region of interest of a target tissue, and obtaining an elastic value measured by strain elastography in a second region of interest of the target tissue, a tissue region corresponding to the first region of interest and a tissue region corresponding to the second region of interest being at least partially overlapped;
determining a first reference value according to an elastic value measured by shear wave elastography in a first reference region in the first region of interest, and determining a second reference value according to an elastic value measured by strain elastography in a second reference region in the second region of interest;
determining a stress value according to the first reference value and the second reference value;
obtaining a target region where the elastic value measured by shear wave elastography is desired to be improved in the first region of interest, the at least partially overlapped tissue region comprising a tissue region corresponding to the target region;
obtaining an elastic value measured by strain elastography corresponding to the target region from the elastic value measured by strain elastography for the second region of interest, and determining a first quantitative elastic value for the target region according to the stress value and the elastic value measured by strain elastography corresponding to the target region;
determining a second quantitative elastic value for the first region of interest according to the elastic value measured by shear wave elastography in the first region of interest and the first quantitative elastic value for the target region; and
displaying the second quantitative elastic value for the first region of interest.

2. The method of claim 1, wherein said determining a second quantitative elastic value for the first region of interest according to the elastic value measured by shear wave elastography for the first region of interest and the first quantitative elastic value for the target region comprises:
replacing the elastic value measured by shear wave elastography for each local point in the target region with the first quantitative elastic value for each local point; the second quantitative elastic value being formed by the first quantitative elastic value for the target region and an elastic value measured by shear wave elastography in a remaining region of the first region of interest excluding the target region.

3. The method of claim 1, wherein said determining a second quantitative elastic value for the first region of interest according to the elastic value measured by shear wave elastography for the first region of interest and the first quantitative elastic value for the target region comprises:
for the target region, calculating a weighted sum of an elastic value measured by shear wave elastography in each local point and a first quantitative elastic value for each local point; the second quantitative elastic value being formed by the weighted sum and an elastic value measured by shear wave elastography in a remaining region of the first region of interest excluding the target region.

4. The method of claim 1, further comprising:
obtaining an elastic value measured by strain elastography in a remaining overlapped region of the at least partially overlapped tissue region excluding the tissue region corresponding to the target region from the elastic value measured by strain elastography for the second region of interest; and
determining a first quantitative elastic value for the remaining overlapped region according to the stress value and the elastic value measured by strain elastography for the remaining overlapped region.

5. The method of claim 4, wherein said determining a second quantitative elastic value for the first region of interest according to the elastic value measured by shear wave elastography for the first region of interest and the first quantitative elastic value for the target region comprises:
calculating a weighted sum of an elastic value measured by shear wave elastography in each local point in the target region and a first quantitative elastic value for said each local point;
calculating a weighted sum of an elastic value measured by shear wave elastography in each local point in the remaining overlapped region and a first quantitative elastic value for said each local point;
when the tissue region corresponding to the second region of interest completely contains the tissue region corresponding to the first region of interest, the second quantitative elastic value being formed by the weighted sum of the elastic value measured by shear wave elastography and the first quantitative elastic value for each local point in the target region and the weighted sum of the elastic value measured by shear wave elastography and the first quantitative elastic value for each local point in the remaining overlapped region; and
when the tissue region corresponding to the first region of interest is partially overlapped with the tissue region corresponding to the second region of interest, the second quantitative elastic value being formed by the weighted sum of the elastic value measured by shear wave elastography and the first quantitative elastic value for each local point in the target region, the weighted sum of the elastic value measured by shear wave elastography and the first quantitative elastic value for each local point in the remaining overlapped region, and an elastic value measured by shear wave elastography in a remaining region of the first region of interest excluding the at least partially overlapped tissue region.

6. The method of claim 4, wherein the target tissue comprises a liver tissue, the target region comprises a focal disease region inside a lesion of the liver tissue, and the remaining overlapped region comprises a diffuse disease region outside the lesion or a non-diseased region outside the lesion of the liver tissue.

7. The method of claim 5, wherein the target tissue comprises a liver tissue, the target region comprises a focal disease region inside a lesion of the liver tissue, and the remaining overlapped region comprises a diffuse disease region outside the lesion or a non-diseased region outside the lesion of the liver tissue.

8. The method of claim 1, wherein said determining a first quantitative elastic value for the target region according to the stress value and the elastic value measured by strain elastography corresponding to the target region comprises:
for each local point in the target region, calculating a quotient between the stress value and an elastic value measured by strain elastography in said local point, and taking the quotient as the first quantitative elastic value for said local point; and
said determining a stress value according to the first reference value and the second reference value comprises:
calculating a product of the first reference value and the second reference value, and taking the product as the stress value.

9. The method of claim 1, wherein said determining a first reference value according to an elastic value measured by shear wave elastography in a first reference region in the first region of interest comprises:
making statistics on a mean of elastic values measured by shear wave elastography for a plurality of local points in the first reference region, and taking the mean as the first reference value; or
determining a median of elastic values measured by shear wave elastography for a plurality of local points in the first reference region, and taking the median as the first reference value.

10. The method of claim 1, wherein said determining a second reference value according to an elastic value measured by strain elastography in a second reference region in the second region of interest comprises:
making statistics on a mean of elastic values measured by strain elastography for a plurality of local points in the second reference region, and taking the mean as the second reference value; or
determining a median of elastic values measured by strain elastography for a plurality of local points in the second reference region, and taking the median as the second reference value.

11. The method of claim 1, wherein the first region of interest and the second region of interest correspond to a same tissue region in the target tissue; and
said obtaining an elastic value measured by shear wave elastography in a first region of interest in a target tissue and obtaining an elastic value measured by strain elastography in a second region of interest of the target tissue comprises:
transmitting acoustic radiation force impulses to the target tissue to generate shear waves propagating in the same tissue region;
transmitting ultrasonic waves to the same tissue region to track the shear waves propagating in the same tissue region, and receiving echoes of the ultrasonic waves to obtain ultrasonic echo data; and
generating the elastic value measured by shear wave elastography for the first region of interest according to the ultrasonic echo data, and generating the elastic value measured by strain elastography for the second region of interest according to the ultrasonic echo data.

12. The method of claim 1, wherein said obtaining an elastic value measured by shear wave elastography in a first region of interest in a target tissue and obtaining an elastic value measured by strain elastography in a second region of interest of the target tissue comprises:
generating first shear waves propagating in the first region of interest;
transmitting first ultrasonic waves to the first region of interest to track the first shear waves propagating in the first region of interest, and receiving echoes of the first ultrasonic waves to obtain first ultrasonic echo signals;
obtaining the elastic value measured by shear wave elastography for the first region of interest according to the first ultrasonic echo signals;
causing a tissue corresponding to the second region of interest to generate displacement or strain;
transmitting second ultrasonic waves at different times to the second region of interest to detect changes in the displacement or strain of the tissue corresponding to the second region of interest, and receiving echoes of the second ultrasonic waves at different times to obtain second ultrasonic echo signals; and
obtaining the elastic value measured by strain elastography for the second region of interest according to the second ultrasonic echo signals.

13. The method of claim 1, further comprising:
making statistics on second quantitative elastic values for a plurality of local points in the target region, and displaying a statistical result therefrom;
and/or,
making statistics on second quantitative elastic values for a plurality of local points in the first region of interest, and displaying a statistical result therefrom;
the statistical result including a median, a mean, a maximum, a minimum, and a standard deviation.

14. The method of claim 1, further comprising:
displaying an ultrasonic image corresponding to the target tissue, and displaying on the ultrasonic image a location of the first region of interest and/or a location of the second region of interest; and/or
displaying the elastic value measured by shear wave elastography for the first region of interest; and/or
displaying the elastic value measured by strain elastography for the second region of interest.

15. The method of claim 1, wherein the elastic value measured by shear wave elastography for the first region of interest is represented as a propagation velocity of shear waves, a Young's modulus or a shear modulus; and the first quantitative elastic value is represented as a propagation velocity of shear waves, a Young's modulus or a shear modulus.

16. The method of claim 1, wherein said displaying the second quantitative elastic value for the first region of interest comprises:
mapping the second quantitative elastic value for the first region of interest into a quantitative elasticity image with a target display effect, and displaying the quantitative elasticity image with the target display effect; the target display effect including any one or more display effects of grayscale, pseudo-color, and color.

17. An ultrasonic imaging apparatus, comprising:
an ultrasonic probe;
a display;
a transmitting circuit configured to excite the ultrasonic probe to transmit ultrasonic waves to a target tissue of an object under examination;
a receiving circuit configured to control the ultrasonic probe to receive echoes of the ultrasonic waves from the target tissue to obtain ultrasonic echo signals; and
a processor configured to process the ultrasonic echo signals to obtain an ultrasonic image of the target tissue, and
obtain an elastic value measured by shear wave elastography in a first region of interest of a target tissue, and obtain an elastic value measured by strain elastography in a second region of interest of the target tissue, a tissue region corresponding to the first region of interest and a tissue region corresponding to the second region of interest being at least partially overlapped;
determine a first reference value according to an elastic value measured by shear wave elastography in a first reference region in the first region of interest, and determine a second reference value according to an elastic value measured by strain elastography in a second reference region in the second region of interest;
determine a stress value according to the first reference value and the second reference value;
obtain a target region where the elastic value measured by shear wave elastography is desired to be improved in the first region of interest, the at least partially overlapped tissue region comprising a tissue region corresponding to the target region;
obtain an elastic value measured by strain elastography corresponding to the target region from the elastic value measured by strain elastography for the second region of interest, and determine a first quantitative elastic value for the target region according to the stress value and the elastic value measured by strain elastography corresponding to the target region;
determine a second quantitative elastic value for the first region of interest according to the elastic value measured by shear wave elastography for the first region of interest and the first quantitative elastic value for the target region; and
control the display to display the second quantitative elastic value for the first region of interest.
